(19) United States
(12) Patent Application Publication
(10) Pub. No.: US 2013/0177599 A1
(43) Pub. Date: Jul. 11, 2013

(54) METHODS AND KITS FOR EXTENDING CONTACT LENS USE

(71) Applicants: Lyle M. BOWMAN, Pleasanton, CA (US); Kamran Hosseini, Hayward, CA (US)

(72) Inventors: Lyle M. BOWMAN, Pleasanton, CA (US); Kamran Hosseini, Hayward, CA (US)

(73) Assignee: INSITE VISION INCORPORATED

(21) Appl. No.: 13/345,087

(22) Filed: Jan. 6, 2012

(51) Int. Cl. A61K 31/78; A61P 27/02; A61K 9/14

(52) U.S. Cl. 424/400; 424/78.04

(57) ABSTRACT

Provided are method and kits useful for extending the wear-time of a contact lens. The method includes applying an amount of an ophthalmically acceptable solution to the contact lens to improve the comfort of the eye when the contact lens is in the eye. The solution includes an aqueous suspension and chitosan. The aqueous suspension includes about 0.1% to about 6.5% by weight of a carboxyl-containing polymer prepared by polymerizing one or more carboxyl-containing monoethylenically unsaturated monomers and less than about 5% by weight of a cross-linking agent. Upon contact with tear fluid, the solution gels to a second viscosity which is greater than the first viscosity. The kit includes contact lenses, an ophthalmically acceptable solution and instructions for applying the solution to improve the comfort of the eye when the contact lens is in the eye.

### BACKGROUND

In recent years contact lens use has increased in part due to the improved vision offered by contact lenses compared to eye glasses, as well as an increase in the variety and availability of contact lens products. Therefore a larger number of individuals are wearing contact lenses and proportionally more people are facing the challenge of discomfort that goes along with the extended wear of contact lenses as a function of time. However, due to the limited water retention properties of contact lenses, wearing contact lenses for long periods of time can cause discomfort and irritation to the eye.

From a clinical point of view, increase in discomfort as a result of contact lens wear often goes hand in hand with microscopic damage to the ocular surface, in particular to the epithelial layer of the cornea. Thus, a need exists for maintaining and improving the comfort of a contact lens in the eye, and thereby extending the wear-time of contact lenses.

### SUMMARY

An aspect of this disclosure is a method of extending the wear-time of a contact lens. The method includes applying an ophthalmically acceptable solution to the contact lens and/or the eye.

In some embodiments, the solution may include a lightly crosslinked carboxyl-containing polymer in an amount sufficient to allow the carboxyl-containing polymer to remain suspended for an extended residence time and hold water in the eye which can help to keep the lens hydrated. The formulation may also contain chitosan as an additive to increase residence time.

In other aspects, embodiments disclosed herein relate to kits for extending the wear-time of a contact lens.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details.

The present disclosure is directed, in part, to methods and kits for extending the wear-time of contact lenses. As used herein, the term “wear-time” means the time a contact lens remains in eye before the user removes the contact lens due to discomfort,. As used herein, the term “discomfort” means any sensation in the eye due to the presence of a contact lens that causes the wearer to have a desire to remove the contact lens.

As used herein, the term “disposable contact lens”, “contact lens”, and “lens” means any lens placed directly onto the front of the eye to correct vision, or to cosmetically change the appearance of the eye.

Examples of the contact lens materials that may be used are: silicone hydrogel, polymethyl methacrylate (PMMA), siloxane acrylates, fluoro-siloxane acrylates, fluoropolymers, and polymers/copolymers of hydroxyethy methacrylate (HEMA), methacrylic acid (MA), n-vinyl pyrolidone (PVP), methyl methacylate (MMA), vinyl acetate (VA), glyceral methacrylate (GMA), acrylic acid (AA), collagen. and mixtures thereof such as polyHEMA, polyHEMA/MA, polyHEMA, polyHEMA/MA, polyHEM/NVP/MMA, polyHEMA/NVP/MMA, polyHEMA/MMA, polyHEMA/GMA, polyHEMA/PC, polyVA, polyHEMA/PVP/MA, polyHEMA/PVA/MA, polyMA/PVP, polyHEMA/PVP/MMA, polyGMA/MMA, polyHEMA/ACR, polyAA/HEMA, polyMMA/AA,

The methods and kits disclosed herein relate to improving comfort in the eye when a contact lens is worn. This is accomplished by providing an ophthalmically acceptable solution for application to the eye or the contact lens. The ophthalmically acceptable solution has rheological properties that may be conducive to delivery into the eye, provide corneal retention, and hydration of a contact lens. The ophthalmically acceptable solution contains about greater than 90% by weight water and holds water in the eye. This ophthalmically acceptable solution has a long ocular residence time and may wash over the surface of the contact lens upon blinking of the eye lid, thus allowing water to be transferred to the contact lens surface.

The ophthalmically acceptable solution may include a combination of an anionic carboxy-containing polymer optionally in conjunction with a substantially smaller amount of a chitosan. The chitosan may be included at a sufficiently low concentration such that the particles of the carboxy-containing polymer remain suspended. When combined with the chitosan, the resulting solution may have a higher viscosity than a solution with the carboxy-containing polymer alone. The ophthalmically acceptable solution may have the property that, when combined with tear fluid, its viscosity increases. The solution may also serve to lubricate and increase the wettability of contact lenses, as well as provide a cushion layer between the lens and the eye.

In an embodiment, the ophthalmically acceptable solution includes an aqueous suspension containing from about 0.1% to about 6.5% by weight, based on the total weight of the suspension, of a carboxyl-containing polymer prepared by polymerizing one or more carboxyl-containing monoethylenically unsaturated monomers and less than about 5% by weight of a crosslinking agent. The weight percentages of monomers are based on the total weight of monomers polymerized. The lightly crosslinked carboxyl-containing polymer has an average particle size of not more than about 5.0 μm in equivalent spherical diameter when dry and approximately 25-28 μm when hydrated at pH 7.4.

The solution may include chitosan in sufficient amount to increase the solution viscosity without the loss of polymer particle suspension, while still allowing the solution to be administered to the eye in drop form. Upon contact of the lower pH solution with higher pH tear fluid, the solution rapidly gels to a greater viscosity and therefore remains on the eye. Alternatively, a high pH formulation may be added to the eye which will reside in the eye for an extended period of time.

As used herein, the term “carboxyl-containing polymer” refers to a polymer that contains a carboxylic acid functional group. This functional group can be substantially ionized, for example, and exist as a carboxylate anion (COO⁻), rendering the polymer negatively charged. An example of a carboxyl-containing polymer that is used herein is lightly crosslinked polycarbophil based polymer.

As used herein the term “lightly crosslinked polymer” encompasses any polymer prepared by suspension or emulsion polymerization having a main polymer backbone comprising at least about 90% by weight of the polymer with a crosslinking agent present in a range from about 0.1% to about 5% by weight of the polymer, including about 0.1%, about 0.2%, about 0.3%, about 0.5%, about 0.6%, about 0.7%, about 0.8%, about 0.9%, about 1.0%, about 1.5%, about 2.0%, about 2.5%, about 3.0%, about 3.5%, about 4.0%, about 4.5%, and about 5.0%, including any fractional amount in between. In some embodiments, the main polymer backbone comprises from about 90% to about 99.9% by weight of the polymer. In some embodiments, the main polymer backbone comprises about 90%, about 91%, about 92%, about 93%, about 94%, about 95%, about 96%, about 97%, about 98%, about 99%, about 99.5%, or about 99.9% by weight of the polymer, including any fractional amount in between. The main polymer backbone can comprise a single monomer unit or can be a copolymer comprising two, three, or any number of monomer units. At least one monomer unit of a main polymer backbone has a functional moiety capable of supporting a charge, such as a carboxyl group, a sulfate group, a phosphate group, and the like. The crosslinking agent may be any difunctional or polyfunctional crosslinking agent.

As used herein “viscosity” refers to a fluid's resistance to flow. The unit of viscosity is dyne second per square centimeter [dyne·s/cm²], or poise [P]. This type of viscosity is also called dynamic viscosity, absolute viscosity, or simple viscosity. This is distinguished from kinematic viscosity which is the ratio of the viscosity of a fluid to its density.

As used herein, “administered to the eye” means that the solution is in the form of an eye drop that can be applied directly to the surface of the contact lens, eye and/or in the cul-de-sac of the eye either prior to applying the lens or after the lens is in the eye. The solution may be applied before the lens is inserted or after the lens is on the eye. The solution may also be applied on the lens, for example, the concave surface of the lens, or maybe used as soaking solution for soaking the lens prior to wearing. The lens may be soaked in the ophthalmically acceptable solution for soaking and disinfecting the lens overnight, upon removal of the lens. Such administration techniques being familiar to persons skilled in the art.

As used herein, “an effective amount” when used in connection with contact lens wear-time is intended to qualify the amount of the solution used in order to provide comfort in the eye when wearing a contact lens so as to extend the wear-time of the contact lens relative to a situation in which no solution is applied to the eye. This amount will achieve the goal of extending wear-time of a contact lens.

In some embodiments, the solution uses a lightly crosslinked polycarbophil based suspension known by the trade name DURASITE®, optionally in conjunction with chitosan added in sufficient amount to increase the solution viscosity, while still allowing the polycarbophil particles to remain suspended. The solution can be in the form of a gel or liquid drops. The lightly crosslinked polycarbophil-based suspension, DURASITE®, is about 0.1 to about 6.5% in some embodiments, and, in other embodiments about 1.0 to about 1.3% by weight based on the total weight of the suspension.

In some embodiments, the solution may also include one or more demulcents. Ophthalmic demulcents are agents, usually water soluble polymers, applied topically to the eye to protect and lubricate mucous membrane surfaces and relieve dryness and irritation. Such demulcents include dextran, cellulose derivatives, polyethylene glycol 400, polyvinylpyrolidone, gelatin, polyols, glycerin, polysorbate 80, propylene glycol, polyvinyl alcohol, povidone (polyvinyl pyrolidone, polysaccaride gels, and Gelrite®. Ophthalmic demulcents or lubricating agents that can be used in ophthalmically acceptable solution may include one or more of those set out in Table 1 below. The amount of ophthalmic demulcent(s) used may generally range from about 0.01% to about 4% by weight, based on the total weight of the formulation. For example, the demulcent, may be used in an amount within the following range:

TABLE 1

| Ophthalmic Demulcent | Amount¹ |
|---|---|
| (a) Cellulose derivatives: | |
| (1) Carboxymethylcellulose sodium | 0.2-2.5% |
| (2) Hydroxymethylcellulose | 0.2-2.5% |
| (3) Hydroxypropylmethylcellulose | 0.2-2.5% |
| (4) Methylcellulose | 0.2-2.5% |
| (b) Dextran 70 | 0.1%² |
| (c) Gelatin | 0.01% |
| (d) Polyols, liquid: | 0.2-1% |
| (1) Glycerin | 0.2-1% |
| (2) polyethyleneglycol 300 (PEG 300) | 0.2-1% |
| (3) polyethyleneglycol 400 (PEG 400) | 0.2-1% |
| (4) Polysorbate 80 | 0.2-1% |
| (5) Propyleneglycol | 0.2-1% |
| (e) Polyvinyl alcohol | 0.1-4% |
| (f) Povidone³ | 0.1-2% |

¹Percents are by weight, based on total weight of formulation
²When used with another polymeric demulcent
³Polyvinvylpyrrolidone

In general, the ophthalmic demulcent or demulcents employed in the ophthalmically acceptable solution may include up to three of the above-listed demulcents and may be used in any amounts from within the above-recited ranges that are compatible with the lightly cross-linked carboxyl-containing polymer. Compatibility in this context means: freedom from the separation of the components of the formulation, whether upon formulation or in storage; the ability of the demulcent-containing gel to be sustained in the presence of tear fluid in the eye for acceptably residence times; and the ability to introduce the demulcent-containing ophthalmically acceptable solution into the eye without provoking more than transient blurring of vision or initial stinging that normally accompanies placing virtually any foreign material in the eye.

In accordance with certain embodiments, the ophthalmically acceptable solution is at a pH of from about 3 to about 8.5 and has an osmolality of from about 10 to about 400 mOsm/kg containing from about 0.1% to about 6.5% by weight, based on the total weight of a suspension of the lightly crosslinked polycarbophil-based polymer DURASITE®, which is prepared by polymerizing one or more carboxyl-containing monoethylenically unsaturated monomers and less than about 5% by weight of a cross-linking agent, such weight percentages of monomers being based on the total weight of monomers polymerized. The lightly crosslinked polycarbophil based suspension DURASITE® can have an initial viscosity of from about 1,000 to about 100,000 centipoises (cps). For example, the viscosity can be in a range from about 1,000 to about 5,000 cps, and in other embodiments from about 5,000 to about 10,000 cps, and in still other embodiments from about 10,000 to about 15,000 cps, and in still further embodiments from about 15,000 to about 20,000 cps, and in yet still further embodiments from about 50,000 to about 100,000 cps, including any values in between these recited values. The lightly crosslinked polycarbophil based suspension DURASITE® has average particle size of not more than about 25 μm hydrated in solution, and in some embodiments, not more than about 15 μm, in equivalent spherical diameter. The lightly crosslinked polycarbophil based suspension DURASITE® is lightly cross-linked to a degree such that although the polymer is administrable in drop form, upon contact of the lower pH suspension with the higher pH tear fluid of the eye, the solution is increases to a substantially greater viscosity than the viscosity of the solution as originally administered in drop form. Accordingly, the resulting more viscous gel can remain in the eye for a prolonged period of time so as maintain the hydration of the contact lens. These properties remain upon addition of the chitosan to the carboxy-containing aqueous suspension. Without being bound by the theory, it is believed that the chitosan increases the viscosity of the base of the lightly crosslinked polycarbophil-based polymer, providing beneficial rheological and mucoadhesive properties. Alternatively, these polymer formulations may be added to the eye or the contact lens at high pH with sufficient viscosity for comfort and extended residence time on the eye.

The carboxy-containing polymer is, in one embodiment, prepared from at least about 50% by weight and in other embodiments from at least about 90% by weight, of one or more carboxyl-containing monoethylenically unsaturated monomers. The lightly crosslinked polycarbophil based suspension DURASITE® can be prepared by suspension or emulsion polymerizing acrylic acid and a non-polyalkenyl polyether difunctional cross-linking agent to a particle size of not more than about 25 μm in one embodiment, and not more than about 15 μm, in equivalent spherical diameter, in other embodiments. In one embodiment, the cross-linking agent is divinyl glycol. In other embodiments, up to about 40% by weight of the carboxyl-containing monoethylenically unsaturated monomers can be replaced by one or more non-carboxyl-containing monoethylenically unsaturated monomers containing only physiologically and ophthalmologically innocuous substituents.

The osmolality, in some embodiments, achieved by using a physiologically and ophthalmologically acceptable salt in an amount of from about 0.01% to about 1% by weight, based on the total weight of the suspensions. Exemplary salts include potassium and sodium chlorides and others as defined above.

A viscosity substantially over 30,000 cps is not useful for drop formulations; when the viscosity is substantially lower than about 1,000 cps, the ability to gel upon contact with tears can be impeded and ocular retention is reduced. The increased gelation upon contact with tears occurs with a pH change when a suspension having a pH of from about 3 to about 7.4 and an osmolality of from about 10 to about 400 mOsm/kg, contacts tear fluid, which has a higher pH of about 7.2 to about 8.0. Without being bound by theory, with an increase in pH, the carboxylic acid (COOH) functional group disassociates into carboxylate anions (COO⁻). Through electrostatic interactions, these carboxylate ions repel each other, causing the polymer to expand. The presence of the trace chitosan in the system can provide additional electrostatic, hydrogen bonding, and possible salt-bridge interactions with the mucins of the ocular mucosa, in addition to providing the initial beneficial viscosity modifying properties to the base solution.

The relationship of cross-linking and particle size can be significant. Because the particles are present in a suspension, the degree of cross-linking is necessarily at a level that avoids substantial dissolution of the polymer. On the other hand, since rapid gelation is achieved at the time of the pH change, the degree of cross-linking is necessarily not so great that gelation is precluded. Moreover, if the polymer particle size is too large, induced swelling can tend to take up voids in the volume between large particles that are in contact with one another, rather than the swelling tending to cause gelation.

In a suspension, particle size can be relevant to comfort. However, in the subject matter of the present disclosure, the small particle size and light cross-linking act synergistically to yield the observed rapid gelation when the pH is raised. Surprisingly, the use of particles greater than 25 μm eliminates the observed gelation when the pH of the solution is increased. Moreover, at the 25 μm size, there is also good eye comfort.

In some embodiments, the particles are not only subject to the upper size limits described above, but also to a narrow particle size distribution. Use of a monodispersion of particles, which aids in good particle packing, yields a maximum increased viscosity upon contact of the suspension with tears and increases eye residence time. At least about 80% in some embodiments, at least about 90% in other embodiments, and at least about 95% in still other embodiments, of the particles are within a no more than about 10 μm dry particle size band of major particle size distribution, and overall (i.e., considering particles both within and outside such band) there should be no more than about 20%, in some embodiments, and no more than about 10%, in other embodiments, and no more than about 5%, in still other embodiments, fines (i.e., particles of a size below 1 μm. In some embodiments, the average particle size is lowered from an upper limit of 10 μm, and to even smaller sizes such as 5 μm, such that the band of major particle size distribution is also narrowed, for example to 3 μm. In some embodiments, sizes for particles within the band of major particle distribution are less than about 5 to 10 μmm, land from about 1 μm to about 5 μm in still other embodiments.

The lightly cross-linked polycarbophil based suspension DURASITE® can be made from a carboxyl-containing monomer or monomers as the sole monoethylenically unsaturated monomer present, together with a cross-linking agent or agents.

The lightly crosslinked polycarbophil based polymer DURASITE® can be prepared by suspension or emulsion polymerizing the monomers, using conventional free radical polymerization catalysts, to a dry particle size of not more than about 5.0 μm in equivalent spherical diameter; e.g., to provide dry polymer particles ranging in size from about 1 to about 10 μm, and in other embodiments from about 3 to about 6 μm, in equivalent spherical diameter. In general, such polymers will range in molecular weight estimated to be about, about 2,000,000,000 to about 4,000,000,000 Daltons.

Aqueous suspensions containing polymer particles prepared by suspension or emulsion polymerization whose average dry particle size is appreciably larger than about 25 μm hydrated particle size in equivalent spherical diameter are less comfortable when administered to the eye than suspensions otherwise identical in composition containing polymer particles whose equivalent spherical diameters are, on the average, below about 25 μm. Moreover, above the average 50 μm size, the advantage of substantially increased viscosity after administration is not realized. It has also been discovered that lightly cross-linked polymers of acrylic acid or the like prepared to a dry particle size appreciably larger than about 50 μm in equivalent spherical diameter and then reduced in size, e.g., by mechanically milling or grinding, to a dry particle size of not more than about 10 μm in equivalent spherical diameter do not work as well as in the inventive ophthalmic solution as polymers made from aqueous suspensions from suspension polymerization because of the particle size distribution.

While not being bound by any theory or mechanism, one possible explanation for the difference of such mechanically milled or ground polymer particles as the sole particulate polymer present is that grinding disrupts the spatial geometry or configuration of the larger than 50 μm lightly cross-linked polymer particles, perhaps by removing uncross-linked branches from polymer chains, by producing particles having sharp edges or protrusions, or by producing ordinarily too broad a range of particle sizes to afford satisfactory delivery system performance. A broad distribution of particle sizes impairs the viscosity-gelation relationship. In any event, such mechanically reduced particles are less easily hydratable in aqueous suspension than particles prepared to the appropriate size by suspension or emulsion polymerization, and also are less able to gel in the eye under the influence of tear fluid to a sufficient extent and are less comfortable once gelled than gels produced in the eye using the aqueous suspensions. However, up to about, 40% by weight, e.g., from about 0.1% to over 20% by weight, based on the total weight of lightly cross-linked particles present, of such milled or ground polymer particles can be admixed with solution or emulsion polymerized polymer particles having dry particle diameters of not more than about 50 μm. Such mixtures also provide satisfactory viscosity levels in the ophthalmically acceptable solution and in the in situ gels formed in the eye coupled with ease and comfort of administration and satisfactory sustained release of the active ingredient to the eye, particularly when such milled or ground polymer particles, in dry form, average from about 0.01 to about 10 μm, and in other embodiments, from about 1 to about 5 μm, in equivalent spherical diameter.

In some embodiments, the particles have a narrow particle size distribution within a 10 μm band of major particle size distribution which contains at least 80%, in other embodiments at least 90%, and in still other embodiments at least 95% of the particles. Also, there is generally no more than about 20%, and in other embodiments no more than about 10%, and in still other embodiments no more than about 5% particles of a size below 1 μm. The presence of large amounts of such fines has been found to inhibit the desired gelation upon eye contact. Apart from that, the use of a monodispersion of particles gives maximum viscosity and an increased eye residence time of the active ingredient in the ophthalmically acceptable solution for a given particle size. Monodisperse particles having a particle size of about 30 □m and below are present in some embodiments. Good particle packing is aided by a narrow particle size distribution.

The ophthalmically acceptable solution can contain amounts of lightly cross-linked polymer particles ranging from about 0.1% to about 6.5% by weight, and in other embodiments from about 0.5% to about 4.5% by weight, based on the total weight of the aqueous suspension. They can be prepared using pure, sterile water, such as deionized or distilled, having no physiologically or ophthalmologically harmful constituents, and are adjusted to a pH of from about 3.0 to about 6.5, and in other embodiments from about 4.0 to about 6.0, using any physiologically and ophthalmologically acceptable pH adjusting acids, bases or buffers, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylaminomethane), or the like and salts and buffers such as citrate/dextrose, sodium bicarbonate, ammonium chloride and mixtures of the aforementioned acids and bases.

Chitosan is obtained by deacetylation of chitin and possesses mucoadhesive properties due to electrostatic interaction between positively charged chitosan ammonium groups and negatively charged mucosal surfaces. Chitosan is a linear polysaccharide composed of randomly distributed β-(1-4)-linked D-glucosamine and N-acetyl-D-glucosamine. Chitosan is available with varying degrees of deacetylation (% DA) and is generally produced in a range from between about 60 to about 100% deacetylation. The amino group in chitosan has a pKa value of about 6.5, thus, chitosan is positively charged and soluble in acidic to neutral solution with a charge density dependent on pH and the % DA-value. Chitosan can enhance the transport of polar drugs across epithelial surfaces, and is considered biocompatible and biodegradable.

In some embodiments, chitosan has a molecular weight in a range from between about 50 kDa to about 100 kDa, including any weights in between, while in other embodiments, chitosan used in the solution has a molecular weight in a range from between about 1,000 to about 3,000 kDa, and any weights in between. As shown in the Examples below, the range between about 1,000 kDa and about 3,000 kDa appears to have a larger impact on viscosity of the solution, even at very small concentrations of the cationic polymer. In order to achieve comparable viscosities with chitosan alone, solutions of chitosan several orders of magnitude more concentrated have been used, for example, from between about 2% to about 4%.

Chitosan is present in an amount ranging from between about 0.01% to about 0.5% when having a molecular weight ranging from about 50 kDa to about 100 kDa. The amount of chitosan can be any amount in between, including about 0.01%, 0.025%, 0.05%. 0.075%, 0.10%, 0.15%, 0.20%, 0.25%, 0.30%, 0.35%, 0.40%, 0.45%, and 0.50% and any amount in between these values. For example, the amount of 1,000 kDa to about 3,000 kDa chitosan can be in a range between about 0.01% and 0.5%, or any amount in between including, for example, 0.01%, 0.015%, 0.020%, 0.025%, 0.030%, 0.035%, 0.040%, 0.045%, 0.05%, 0.1%, 0.15%, 0.20%, 0.25%, 0.30%, 0.35%, 0.40%, 0.45%, and 0.50%.

When formulating the ophthalmically acceptable solution, their osmolality can be adjusted to from about 10 mOsm/kg to about 400 mOsm/kg, and in other embodiments, from about 100 to about 300 mOsm/kg, using appropriate amounts of physiologically and ophthalmologically acceptable salts. Sodium chloride can be used as an osmolality adjusting agent to adjust the osmolality of the aqueous suspension to approximate that of physiologic fluid. The amounts of sodium chloride ranging from about 0.01% to about 1% by weight, and in other embodiments from about 0.05% to about 0.45% by weight, based on the total weight of the aqueous suspension, will give osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like can also be used in addition to or instead of sodium chloride to achieve osmolalities within the above-stated ranges.

The amounts of lightly cross-linked carboxy-containing polymer particles, cationic polymer, the pH, and the osmolality chosen from within the above-stated ranges can be correlated with each other and with the degree of cross-linking to give aqueous suspensions having viscosities ranging from about 1,000 to about 30,000 cps, and in other embodiments from about 5,000 to about 20,000 cps, as measured at room temperature (about 25° C.) using a Brookfield Digital LVT Viscometer equipped with a number 25 spindle and a 13R small sample adapter at 12 rpm. The correlations of those parameters are also such that the suspensions will gel on contact with tear fluid to give gels having viscosities estimated to range from about 75,000 to about 500,000 cps, e.g., from about 200,000 to about 300,000 cps, measured as above, depending on pH as observed, for example, from pH-viscosity curves. This effect is noted by observing a more viscous drop on the eye as a set cast. The cast, after setting, can be easily removed. Alternatively, the viscosity can be from about 1000 to about 5000 cps as measured with a Brookfield cone and plate viscometer DV-II+ with the spindle no. CP-52 at 6 rpm.

In some embodiments, the viscosity is in a range from about 1,000 to about 30,000 cps, and in other embodiment from about 5,000 to about 20,000 cps. In yet other embodiments, the viscosity is in a range from about 10,000 to about 15,000 cps. The viscosity range can also be between about 1,000 and 5,000 cps, including 1,000, 1,500, 2,000, 2,500, 3,000, 3,500, 4,000, 4500, and 5,000 cps and all values in between. The viscosity range can also be between about 5,000 to about 10,000 cps, including 5,000, 5,500, 6,000, 6,500, 7,000, 7,500, 8,000, 8,500, 9,000, 9,500, and 10,000 cps and all values in between. The viscosity range can also be between about 10,000 to about 15,000 cps, including 10,000, 10,500, 11,000, 11,500, 12,000, 12,500, 13,000, 13,500, 14,000, 14,500, and 15,000 cps and all values in between. The viscosity range can also be between about 15,000 to about 20,000 cps, including 15,000, 15,500, 16,000, 16,500, 17,000, 17,500, 18,000, 18,500, 19,000, 19,500, and 20,000 cps and all values in between. The viscosity range can also be between about 20,000 to about 30,000 cps, including 20,000, 21,000, 22,000, 23,000, 24,000, 25,000, 26,000, 27,000, 28,000, 29,000, and 30,000 cps and all values in between. In some embodiments, the ophthalmically acceptable solution can include a thickening agent or viscosfier that modulates the viscosity of the solution. These include, without limitation, polyethylene glycols, polyvinyl alcohol, polyacrylic acid, polyethylene oxide, and poloxamers.

The ophthalmically acceptable solution can be packaged in preservative-free, reclosable containers or kits. In addition, a contact lens may be pre-soaked in the ophthalmically acceptable solution and sealed in a container.

In those ophthalmically acceptable solutions where preservatives are to be included, suitable preservatives are chlorobutanol, Polyquat, benzalkonium chloride, cetyl bromide, benzethonium chloride, cetyl pyridinium chloride, benzyl bromide, phenylmercury nitrate, phenylmercury acetate, thimerosal, merthiolate, acetate and phenylmercury borate, chlorhexidine, polymyxin B sulphate, methyl and propyl parabens, phenylethyl alcohol, quaternary ammonium chloride, sodium benzoate, sodium proprionate, sorbic acid, and sodium perborate. In particular embodiments, the preservative includes benzalkonium chloride.

In some embodiments, the preservative is present in a range from about 0.001 to about 0.02% by weight. The preservative can be present at about 0.001, 0.002, 0.003, 0.004, 0.005% and any amount in between these amounts. In particular, the present methods and kits have the benefit of substantial reduction in the use of a bactericidal component. Thus, in some embodiments, suspension has less than about 0.01% of a preservative with bactericidal activity in one embodiment, and less than about0.01%, 0.009%, 0.008%, 0.007%, 0.006%, 0.005%, 0.004%, 0.003%, or 0.002%, in other embodiments.

In some embodiments, the ophthalmically acceptable solution may include a wetting agent. Such wetting agents include, for example, Poloxamer 407, a triblock copolymer consisting of a central hydrophobic block of polypropylene glycol flanked by two hydrophilic blocks of polyethylene glycol. Other wetting agents that can be used include carboxymethylcellulose, hydroxypropyl methylcellulose, glycerin, mannitol, polyvinyl alcohol, Octoxynol 40 and hydroxyethylcellulose.

In some embodiments a kit may include: (a) the ophthalmically acceptable solution stored in a preservative-free unit-dose containers; (b) a supply of contact lenses stored in a sealed single use container and may be soaked in the ophthalmically acceptable solution; (c) instructions for applying the solution and contact lenses; and (d) a reclosable, reuseable container for storing the contact lenses in the ophthalmically acceptable solution.

The kit may further include information on the use of the ophthalmically acceptable solution and lens or a pre-recorded media device which, e.g., provides information on the use of the present method.

The kit may also include a container for storing the components of the kit. The container can be, for example, a bag, box, envelope or any other container suitable for use. In some embodiments, the container is large enough to accommodate each component. However, in some cases, it can be desirable to have a smaller container which is large enough to carry only some of the components.

### EXAMPLES OF APPLICATION

### 1. Applying Solution to Contact Lens Outside of the Eye

After removal of a contact lens from the eye, the lens may be rinsed with a cleaning, disinfecting and/or storing liquid. Various cleaning, disinfecting and storing liquids have been described in the art. The contact lens may then be stored in a container and soaked in the ophthalmically acceptable solution and disinfecting/storing liquid for at least three hours until the lens is again placed in the eye sufficient to cover the lens in the container. The lens is rinsed with saline and an amount of the ophthalmically acceptable solution (for example 25-35 μl) may then be placed in drop form one or both sides of the contact lens surface(s). The contact lens may then be placed in the eye.

### 2. Applying Solution to Contact Lens When Lens is in the Eye

While the contact lens is in eye, an amount of the ophthalmically acceptable solution (for example 25-35 μl drop) may be placed in drop form either directly on the outside surface of the lens, or directly to the eye or in the cul-de-sac.

### 3. Applying Solution to the Eye Prior to Placing Lens in the Eye

Prior to placing a contact lens is in eye, an amount of the ophthalmically acceptable solution (for example 25-35 μl drop) may be placed in the eye and then the lens is placed in the eye.

### 4. Providing Solution in Sealed Contact Lens Packaging Prior to First Time Use

The contact lens after manufacture may be stored in the ophthalmically acceptable solution and stored or shipped. This contact lens in this solution may be directly applied to the eye for the first time. The contact lens in a sealed contact lens package can be directly taken from the package and placed in the eye.

### 5. Providing Solution for Soaking and Disinfecting Contact Lens

After removal of the contact lens from the eye and prior to re-application of the contact lens to the eye, an amount of the ophthalmically acceptable solution acceptable for disinfection may be placed in the contact lens container with the lens to soak and disinfect the lens.

It is understood that modifications which do not substantially affect the activity of the various embodiments of this

Formulations

TABLE 2

| Component | 1 | 3 | 3 | 4 | 5 | 6 | 7 | 8 | 9 | 10 |
|---|---|---|---|---|---|---|---|---|---|---|
| Polycarbophil | 0.9 | 0.9 | 0.9 | 0.8 | 0.9 | 0.9 | 0.9 | 0.9 | 0.9 | 0.9 |
| PEG-400 | — | — | — | 0.2 | 0.2 | 0.2 | 0.2 | 0.2 | — | — |
| Sodium Chloride | 0.6 | 0.6 | 0.6 | 0.4 | 0.4 | 0.4 | 0.04 | 0.045 | 0.6 | 0.6 |
| Poloxamer 407 | — | — | — | — | 0.2 | 0.2 | 0.2 | — | — | — |
| Sodium Edetate | 0.1 | 0.1 | 0.025 | 0.1 | 0.1 | 0.1 | 0.1 | 0.1 | 0.1 | 0.1 |
| Mannitol | — | — | — | 1.0 | — | — | — | — | — | — |
| Glycerin | — | — | — | 1.0 | 1.0 | 1.0 | 1.0 | 1.0 | | |
| Sodium | qs to | qs to | qs to | qs to | qs to | qs to | qs to | qs to | qs to | qs to |
| Hydroxide | ph 6.3 | ph 8.3 | ph 6.3 | ph 6.3 | ph 7.4 | ph 7.4 | ph 8.3 | ph 7.4 | ph 7.4 | ph 6.8 |
| Benzalkonium | 0.001 | 0.001 | — | 0.001 | 0.001 | — | — | — | — | — |
| Chloride | | | | | | | | | | |
| Sodium Perborate | — | — | — | — | — | 0.1 | — | 0.1 | 0.25 | — |
| Dequest | — | — | — | — | — | 0.1 | — | 0.1 | 0.1 | — |
| Sodium Borate | — | — | — | — | — | — | 0.51 | — | — | — |
| Boric Acid | — | — | — | — | — | — | 0.49 | — | — | — |
| Sorbic Acid | — | — | — | — | — | — | — | — | — | 0.2 |

The samples in examples 1-10 are made by adding polycarbophil, sodium chloride and edetate to water by stirring for 0.5 hours. The solution is then sterilized at 121° C. for 45 minutes and cooled to room temperature. The following ingredients if present such as mannitol, poloxamer, PEG-400, glycerin, are dissolved in water and added to the batch by sterile addition through a 0.2 μm filter. The following items if present such as borate buffer, benzalkonium chloride or sorbic acid or perborate/dequest are dissolved in water and added by sterile filtration while mixing the formulation. Sodium hydroxide is added by sterile addition to adjust the pH to the desired pH. Formulation number 9 shown above can be used to store lenses a minimum of 3 hours to disinfect the lens after wearing due to the perborate disinfectant.

Those skilled in the art will readily appreciate that the specific examples and studies detailed above are only illustrative. The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method for extending the comfortable wear-time of a contact lens comprising:
applying an ophthalmically acceptable solution to a surface of the contact lens or the eye; and
placing the contact lens in the eye,
wherein the ophthalmically acceptable solution comprises, an aqueous polymeric suspension having a first viscosity, the suspension comprising from about 0.1% to about 6.5% by weight, based on the total weight of the suspension, of a carboxyl-containing polymer prepared by polymerizing one or more carboxyl-containing monoethylenically unsaturated monomers and less than about 5% by weight of a cross-linking agent, the weight percentages of monomers being based on the total weight of monomers polymerized, the carboxyl-containing polymer having average particle size of not more than about 25 μm in equivalent hydrated spherical diameter.

2. The method of claim 1, wherein the carboxyl-containing polymer is polycarbophil.

3. The method of claim 1, wherein the ophthalmically acceptable solution further comprises a sufficient amount of a second polymer allowing said carboxyl-containing polymer to remain suspended,
wherein upon contact with tear fluid, said solution gels to a second viscosity which is greater than the first viscosity.

4. The method of claim 3, wherein the second polymer is chitosan.

5. The method of claim 4, wherein the chitosan is present in a range from between about 0.01% to about 0.05% by weight of the solution.

6. The method of claim 1, wherein the ophthalmically acceptable solution further comprises a sufficient amount of a demulcent.

7. The method of claim 6, wherein the demulcent is selected from the group consisting of dextran, cellulose derivatives, polyethylene glycol 400, polyvinylpyrolidone, gelatin, polyols, glycerin, polysorbate 80, propylene glycol, polyvinyl alcohol, polyvinyl pyrrolidone polysaccaride gels and Gelrite®

8. The method of claim 1, further comprising a step of soaking the contact lens in the solution for at minimum of 3 hours upon removal of the contact lens from the eye.

9. The method of claim 8, further comprising a step of rinsing the contact lens with disinfectant prior to soaking the contact lens in the solution.

10. The method of claim 1, further comprising a step of applying the solution to the eye.

11. A kit for rehydrating the eye comprising:
one or more lenses for application directly to the eye; and
an ophthalmically acceptable solution, stored in a reclosable container, wherein the ophthalmically acceptable solution comprises:
an aqueous suspension having a first viscosity, said suspension comprising from about 0.1% to about 6.5% by weight, based on the total weight of the suspension, of a carboxyl-containing polymer prepared by polymerizing one or more carboxyl-containing monoethylenically unsaturated monomers and less than about 5% by weight of a cross-linking agent, said weight percentages of monomers being based on the total weight of monomers polymerized, said carboxyl-containing polymer having average particle size of not more than about 25 μm in equivalent hydrated spherical diameter.

12. The kit of claim 11, wherein the ophthalmically acceptable solution further comprises a sufficient amount of a demulcent.

13. The kit of claim 12, wherein the demulcent is selected from the group consisting of dextran, cellulose derivatives, polyethylene glycol 400, polyvinylpyrolidone, gelatin, polyols, glycerin, polysorbate 80, propylene glycol, polyvinyl alcohol, polyvinyl pyrrolidone polysaccaride gels and Gelrite®

14. The kit of claim 12, where in the solution contains a disinfectant.

15. A method comprising the steps of:
applying an ophthalmically acceptable solution to the eye; and
placing a contact lens in an eye,
wherein the ophthalmically acceptable solution comprises, an aqueous polymeric suspension having a first viscosity, the suspension comprising from about 0.1% to about 6.5% by weight, based on the total weight of the suspension, of a carboxyl-containing polymer prepared by polymerizing one or more carboxyl-containing monoethylenically unsaturated monomers and less than about 5% by weight of a cross-linking agent, the weight percentages of monomers being based on the total weight of monomers polymerized, the carboxyl-containing polymer having average particle size of not more than about 25 μm in equivalent hydrated spherical diameter.

16. The method of claim 15, wherein the carboxyl-containing polymer is polycarbophil.

17. The method of claim 15, wherein the ophthalmically acceptable solution further comprises a sufficient amount of a second polymer allowing said carboxyl-containing polymer to remain suspended,
wherein upon contact with tear fluid, said solution gels to a second viscosity which is greater than the first viscosity.

18. The method of claim 15, wherein the second polymer is chitosan.

19. The solution of claim 18, wherein the chitosan is present in a range from between about 0.01% to about 0.05% by weight of the solution.

20. The method of claim 15, wherein the ophthalmically acceptable solution further comprises a sufficient amount of a demulcent.

21. The method of claim 20, wherein the demulcent is selected from the group consisting of dextran, cellulose derivatives, polyethylene glycol 400, polyvinylpyrolidone, gelatin, polyols, glycerin, polysorbate 80, propylene glycol, polyvinyl alcohol, polyvinyl pyrrolidone polysaccaride gels and Gelrite®
